(19) United States
(12) Patent Application Publication
(10) Pub. No.: US 2003/0082631 A1
(43) Pub. Date: May 1, 2003

(54) Compounds modulating PPAR-gamma

(76) Inventors: Anna-Lena Gustavsson, Stockholm (SE); Stefan Svensson, Stockholm (SE); Jonas Uppenberg, Bromma (SE)

Correspondence Address: FISH & RICHARDSON PC, 225 FRANKLIN ST, BOSTON, MA, 02110, US

(21) Appl. No.: 10/190,264

(22) Filed: Jul. 3, 2002

Related U.S. Application Data
(60) Provisional application No. 60/304,706, filed on Jul. 11, 2001.

(30) Foreign Application Priority Data
Jul. 3, 2001 (SE) 0102384-5

(51) Int. Cl. G01N 33/53; A61K 31/24; A61K 31/381

(52) U.S. Cl. 435/7.1; 514/535; 514/438

(57) ABSTRACT

The present invention relates to a method for identifying compounds capable of binding the ligand binding domain of peroxisome proliferator-activated receptor gamma (PPARγ), and selectively modulating the activity of PPARγ. The said method includes providing compounds that fit spatially and preferentially into a PPARγ ligand binding domain having the pharmacophoric features shown in Table I in the patent specification.

### RELATED APPLICATIONS

This application claims priority to Swedish application number 0102384-5, filed on Jul. 3, 2001, and U.S. provisional application No. 60/304,706, filed on Jul. 11, 2001, the contents of which are incorporated herein by reference.

### TECHNICAL FIELD

The present invention relates to methods for identifying compounds capable of binding the ligand binding domain of peroxisome proliferator-activated receptor gamma (PPARγ), and selectively modulating the activity of PPARγ. The invention also relates to compounds identified by the said method.

### BACKGROUND ART

The peroxisome proliferator-activated receptors (PPARs) are members of the nuclear hormone receptor subfamily of transcription factors. PPARs form heterodimers with retinoid X receptors (RXRs) and these heterodimers regulate transcription of various genes. PPARs may be involved in diseases such as diabetes, obesity, atherosclerosis and cancer. This, together with the fact that PPAR activity can be modulated by drugs such as thiazolidinediones and fibrates, has instigated a huge research effort into PPARs (Desvergene, B. & Wahli, W. (1999) Endocr. Rev. 20, 649-688). For further reviews on PPARs and their medical significance, see e.g. Berger & Moller (2002) Annu. Rev. Med. 53: 409-435; Kersten, S. et al. (2000) Nature 405:421-424; Willson, T. M. et al. (2000) J. Med. Chem. 43:527-550; Vamecq, J. et al. (1999) Lancet 354:141-148.

Three PPAR isotypes have been identified: α, β (also called δ and NUC1) and γ. PPARα is expressed most in brown adipose tissue and liver, then kidney, heart and skeletal muscle. PPARγ (GenBank Accession No. X90563) is mainly expressed in adipose tissue, and to a lesser extent in colon, the immune system and the retina. Whereas PPARα operates in the catabolism of fatty acids in the liver, PPARγ influences the storage of fatty acids in the adipose tissue. With the C/EBP transcription factors, PPARγ is part of the adipocyte differentiation program that induces the maturation of pre-adipocytes into fat cells (Rosen, E. D. et al. (1999) Mol. Cell 4, 611-617). Most of the PPARγ target genes in adipose tissue are directly implicated in lipogenic pathways, including lipoprotein lipase (LPL), adipocyte fatty acid binding protein (A-FABP or aP2), acyl-CoA synthase and fatty acid transport protein (FATP). Lowell (Cell 99: 239-242; 1999) reviewed the role of PPARγ in adipogenesis.

In adipose tissue, the amounts of sterol response element binding protein 1 (SREBP1) and PPARγ are elevated, probably because of regulation by insulin (Rieusset, J. et al. (1999) Diabetes 48, 699-705). PPARγ is a direct target gene of SREBP1 (Fajas, L. et al. (1999) Mol. Cell. Biol. 19, 5495-5503), which emphasizes the cooperative and additive functions between these two types of receptor. In addition, SREBP1 may be involved in producing an endogenous ligand (probably fatty acid) for PPARγ. The overall effect is stimulation of the uptake of glucose and fatty acids, and their subsequent conversion to triglycerides.

Metabolic disorders such as hyperlipidaemia, atherosclerosis, diabetes and obesity rarely occur in isolation, but are usually part of a complex phenotype of metabolic abnormalities called syndrome X. Synthetic agonists for both PPARα (fibrates) and PPARγ (thiazolidinediones; TZDs) are useful in the treatment of the diseases that are part of this syndrome. Synthetic PPARγ ligands are used for their potent antidiabetic effects. In the United States, three TZDs, troglitazone (Rezulin), rosiglitazone (Avandia) and pioglitazone (Actos), are approved for use in type II diabetic patients. They bind PPARγ with moderate (troglitazone) to high (rosiglitazone) affinity, so it is believed that their hypoglycaemic effect is exerted by activating PPARγ.

The X-ray crystal structure of apo-PPARγ LBD was reported by Nolte et al. (1998; Nature 395: 137-143) and Uppenberg et al. (1998; J. Biol. Chem. 273: 31108-31112). The structure revealed a total of 12 helices and a small β-sheet of four strands. Helix 12 was predicted to cover the predicted LBD (ligand-binding domain) pocket, which could be divided into two interconnected cavities, both of which extend into a wide surface accessible groove parallel to helix 3. Nolte et al. also reported the structure of holo-PPARγ LBD in complex with rosiglitazone. This structure identified the amino acid side chains of His323, His449 and Tyr473 as being important residues for receptor-ligand interactions and it was suggested that the binding of ligands to these residues would be critical for coactivator binding and transcriptional activation of the target gene. The structural basis for PPARγ activation by ligands is reviewed by Willson et al. (2001) Annu. Rev. Biochem. 70: 341-367. More recent data suggest that the nature of PPARγ ligands can influence the receptor binding preferences for different coactivators. As a consequence different ligands show different physiological effects (Rochi et al. (2001) Mol. Cell 8:737-747). This opens up the possibility of developing “selective PPAR modulators” with tissue specific activities (Rangwala & Lazar (2002) Sci STKE Vol. 2002 (121): PE9), in analogy with the much-studied “selective estrogen receptor modulators” (SERMs) (Shang et al. (2002) Science 295:2465-2468).

### BRIEF DESCRIPTION OF THE DRAWINGS

FIG. 1 illustrates the crystal structure of the compound lithium 2-[(2,4-dichlorobenzoyl)amino]-5-(2-thienylmethoxy)benzoate in complex with the PPARγ ligand binding domain. The atomic coordinates of the crystal structure are those in TABLE 1.

FIG. 2 illustrates the new identified pharmacophore model represented by the compound lithium 2-[(2,4-dichlorobenzoyl)amino]-5-(2-thienylmethoxy)benzoate in its bioactive conformation.

### DISCLOSURE OF THE INVENTION

The present invention relates to compounds modulating PPARγ by a previously unknown binding mode. Co-crystal structures of PPARγ and ligands to PPARγ have been determined by X-ray diffraction. The identified binding mode of the compounds reveals a novel pharmacophoric pattern for PPARγ ligands with the potential to selectively modulate the binding of coactivators and activate gene transcription. The invention comprises the use of this pharmacophore model and the X-ray structure as design tools for new classes of PPARγ modulators. These classes of modulators are predicted to be useful in the treatment of metabolic diseases, e.g. type II diabetes. The amino acid sequence of PPARγ is shown below (SEQ ID NO: 1) and the X-ray structure atomic coordinates are provided in, e.g., Uppenberg et al. (1998) J. Bio. Chem. 273: 31108-31112. Also see Protein Data Bank (http://www.rcsb.org/pdb/), code 3prg. Those of skill in the art will understand that a set of structure coordinates for a protein (e.g., PPARγ), is a relative set of points that define a shape in three dimensions. Thus, it is possible that an entirely different set of coordinates could define a similar or identical shape. Moreover, slight variations in the individual coordinates will have little effect on overall shape.

| Met Thr Met Val Asp Thr Glu Met Pro Phe Trp Pro Thr Asn Phe Gly | (SEQ ID NO:1) |
|---|---|
| 1 5 10 15 | |
| Ile Ser Ser Val Asp Leu Ser Val Met Glu Asp His Ser His Ser Phe | |
| 20 25 30 | |
| Asp Ile Lys Pro Phe Thr Thr Val Asp Phe Ser Ser Ile Ser Thr Pro | |
| 35 40 45 | |
| His Tyr Glu Asp Ile Pro Phe Thr Arg Thr Asp Pro Val Val Ala Asp | |
| 50 55 60 | |
| Tyr Lys Tyr Asp Leu Lys Leu Gln Glu Tyr Gln Ser Ala Ile Lys Val | |
| 65 70 75 80 | |
| Glu Pro Ala Ser Pro Pro Tyr Tyr Ser Glu Lys Thr Gln Leu Tyr Asn | |
| 85 90 95 | |
| Lys Pro His Glu Glu Pro Ser Asn Ser Leu Met Ala Ile Glu Cys Arg | |
| 100 105 110 | |
| Val Cys Gly Asp Lys Ala Ser Gly Phe His Tyr Gly Val His Ala Cys | |
| 115 120 125 | |
| Glu Gly Cys Lys Gly Phe Phe Arg Arg Thr Ile Arg Leu Lys Leu Ile | |
| 130 135 140 | |
| Tyr Asp Arg Cys Asp Leu Asn Cys Arg Ile His Lys Lys Ser Arg Asn | |
| 145 150 155 160 | |
| Lys Cys Gln Tyr Cys Arg Phe Gln Lys Cys Leu Ala Val Gly Met Ser | |
| 165 170 175 | |
| His Asn Ala Ile Arg Phe Gly Arg Met Pro Gln Ala Gln Lys Glu Lys | |
| 180 185 190 | |
| Leu Leu Ala Glu Ile Ser Ser Asp Ile Asp Gln Leu Asn Pro Gln Ser | |
| 195 200 205 | |
| Ala Asp Leu Arg Ala Leu Ala Lys His Leu Tyr Asp Ser Tyr Ile Lys | |
| 210 215 220 | |
| Ser Phe Pro Leu Thr Lys Ala Lys Ala Arg Ala Ile Leu Thr Gly Lys | |
| 225 230 235 240 | |
| Thr Thr Asp Lys Ser Pro Phe Val Ile Tyr Asp Met Asn Ser Leu Met | |
| 245 250 255 | |
| Met Gly Glu Asp Lys Ile Lys Phe Lys His Ile Thr Pro Leu Gln Glu | |
| 260 265 270 | |
| Gln Ser Lys Gln Val Ala Ile Arg Ile Phe Gln Gly Cys Gln Phe Arg | |
| 275 280 285 | |
| Ser Val Glu Ala Val Gln Glu Ile Thr Glu Tyr Ala Lys Ser Ile Pro | |
| 290 295 300 | |
| Gly Phe Val Asn Leu Asp Leu Asn Asp Gln Val Thr Leu Leu Lys Tyr | |
| 305 310 315 320 | |
| Gly Val His Glu Ile Ile Tyr Thr Met Leu Ala Ser Leu Met Asn Lys | |
| 325 330 335 | |
| Asp Gly Val Leu Ile Ser Glu Gly Gln Gly Phe Met Thr Arg Glu Phe | |
| 340 345 350 | |
| Leu Lys Ser Leu Arg Lys Pro Phe Gly Asp Phe Met Glu Pro Lys Phe | |
| 355 360 365 | |
| Glu Phe Ala Val Lys Phe Asn Ala Leu Glu Leu Asp Asp Ser Asp Leu | |
| 370 375 380 | |
| Ala Ile Phe Ile Ala Val Ile Ile Leu Ser Gly Asp Arg Pro Gly Leu | |
| 385 390 395 400 | |
| Leu Asn Val Lys Pro Ile Glu Asp Ile Gln Asp Asn Leu Leu Gln Ala | |
| 405 410 415 | |
| Leu Glu Leu Gln Leu Lys Leu Asn His Pro Glu Ser Ser Gln Leu Phe | |
| 420 425 430 | |
| Ala Lys Leu Leu Gln Lys Met Thr Asp Leu Arg Gln Ile Val Thr Glu | |
| 435 440 445 | |
| His Val Gln Leu Leu Gln Val Ile Lys Lys Thr Glu Thr Asp Met Ser | |
| 450 455 460 | |
| Leu His Pro Leu Leu Gln Glu Ile Tyr Lys Asp Leu Tyr | |
| 465 470 475 | |

Consequently, in a first aspect this invention provides a method for identifying a compound capable of selectively modulating, in particular agonizing, the activity of PPARγ, said method comprising:

(i) providing test compounds that fit spatially and preferentially (contain interactions described below) into a PPARγ ligand binding domain;

(ii) screening said test compounds for binding to the PPARγ ligand binding domain; and

(ii) identifying a test compound that selectively modulates the activity of PPARγ;

wherein said compound comprises:

(a) a benzoate group wherein the aromatic ring is capable of interacting with the side chains of Ile341 and Cys285 of SEQ ID NO: 1 and the back bone atoms of Gly284 and Cys285 of SEQ ID NO: 1;

(b) a carboxylate group bound to the benzoate group of (a), said carboxylate moiety being capable of interacting, by polar interaction, with the backbone amide nitrogen of residue Ser342 of SEQ ID NO: 1; preferably, the carboxylate group is stabilized by an internal hydrogen bond to an amide nitrogen on the ligand; and

(c) an aromatic group bound by an amide group to the benzoate group of (a), the said aromatic group being located in a hydrophobic region and being capable of interacting with the side chains of Leu330, Ile326, Arg288, Leu333 and Met329 of SEQ ID NO:1.

Assays to determine if a compound modulates (e.g., stimulates or inhibits) the activity of PPARγ are well known in the art and are also illustrated in the examples below.

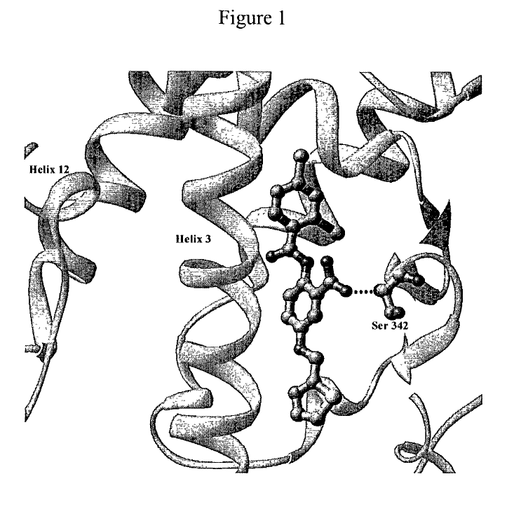

The term “ligand binding domain,” as used herein, refers to a region of PPARγ protein, that, as a result of its shape, favorably binds to a ligand (e.g., a peptide or an organic molecule). The ligand binding domain includes amino acids Gly284, Cys285, Arg288, Ile326, Met329, Leu330, Leu333, Ile341, and Ser342 of SEQ ID NO: 1, and its shape can be defined by atomic coordinates of these amino acids according to FIG. 1 or Table 1.

The term “coordinates” refers to three-dimensional atomic coordinates derived from mathematical equations related to the experimentally measured intensities obtained upon diffraction of a mono- or polychromatic beam of X-rays by the atoms (scattering centers) of a protein or protein-ligand complex in crystal form. The diffraction data may be used to calculate an electron density map of the repeating unit of the crystal. The electron density maps can be used to establish the positions of the individual atoms within the unit cell of the crystal. Alternatively, computer programs such as XPLOR can be used to establish and refine the positions of individual atoms. Those of skill in the art understand that a set of structure coordinates determined by X-ray crystallography is not without error. For the purposes of this invention, any set of structure coordinates for a PPARγ, that have a root mean square deviation of equivalent protein backbone atoms (N, Cα, C and O) of less than about 1.50 Å, or alternatively less than about 1.00 Å when superimposed, using backbone atoms, on the structure coordinates listed herein shall be considered identical and within the scope of the invention.

The term “unit cell” refers to a basic parallelipiped shaped block. The entire volume of a crystal may be constructed by regular assembly of such blocks. Each unit cell comprises a complete representation of the unit of pattern, the repetition of which builds up the crystal.

The term “complex” refers to a protein in covalent or non-covalent association with a ligand, such ligand including, for example, a chemical entity, compound, or inhibitor, candidate drug, and the like. The term “association” refers to a condition of proximity between the ligand and the protein, or their respective portions thereof, in any appropriate physicochemical interaction.

The term “bind” or “binding” refers to non-covalent molecular interactions that include hydrogen bonding, van der Waals interactions, hydrophobic interactions, and electrostatic interactions.

The term “selective modulating” refers to those compounds modulating the activity of PPARγ more than the other proteins, such as modulating the activity of PPARγ at least 20% (e.g., 30%, 50%, 80%, or 100%) more than the others.

In a preferred aspect of the invention, the PPARγ ligand binding domain is based on a structural model of PPARγ bound to the compound lithium 2-[(2,4-dichlorobenzoyl)amino]-5-(2-thienylmethoxy)benzoate, and has the pharmacophoric features shown in TABLE 1 or 2. The test compounds can e.g. be provided by structure-based design, or by virtual screening of compound databases using the pharmacophore described above as search pattern.

The structure-based design method is a method for optimizing interactions between a protein, e.g., PPARγ, and a compound (e.g., a test compound, a compound of formula I) by determining and evaluating the three-dimensional structure of successive sets of protein/compound complexes. The method may incorporate computer-assisted drug design (CADD) techniques, known in the art and examples of which are delineated herein. It may begin by visual inspection of, e.g., a PPARγ ligand binding domain on the computer screen based on the atomic coordinates in FIG. 1 or other coordinates which define a similar shape. Selected fragments of a compound may then be positioned in a variety of orientations, or docked, within that binding domain as defined supra. Docking may be accomplished using computer software, followed by energy minimization and molecular dynamics with standard molecular mechanics force fields. Instead of proceeding to build a compound in a step-wise fashion one fragment at a time as described above, a compound may be designed as a whole or “de novo” using either an empty binding site or optionally including some portion(s) of a known compound (e.g., a known binding ligand). The virtual screening is computational screening of small molecule databases for compounds that can bind in whole, or in part, to a binding domain (e.g., a PPARγ ligand binding domain). In this screening, the quality of fit of such compounds to the binding domain may be judged either by shape complementarity or by estimated interaction energy.

Included in the invention are compounds identified by the methods as described above. In a preferred aspect, such a compound has the formula I

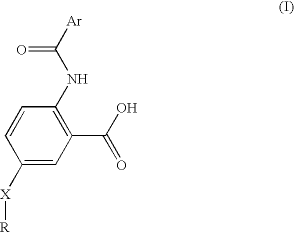

or is a pharmaceutically acceptable salt or a prodrug form thereof, wherein

Ar is a 5- or 6-membered aromatic group or a fused aromatic ring system, e.g. phenyl, imidazole or naphthyl, substituted with a group expanding into the unoccupied part of the ligand binding pocket and making hydrogen bonding interactions with one or more of the side chain of Tyr473, His323 and His449 of SEQ ID NO: 1; in this context the term “unoccupied part of the ligand binding pocket” shall mean the region of the binding pocket that is delimited by the side chains of Phe282, Cys285, His323, Tyr327, Phe363, Met364, Lys367, His449, Leu469 and Tyr473 of SEQ ID NO: 1;

X is

a bond, or

a heteroalkyl chain comprising from 1 to 4 carbon atoms and from 1 to 4 heteroatoms, or

a formula

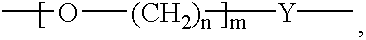

wherein m is 0, 1, or 2,

n is 0, 1, 2, or 3, and

Y is a bond, O, S, NH, NHSO₂, NHC(O)NH, or CH═CH; and

R is an optionally substituted aryl or heteroaryl group.

Preferred compounds of the formula I include those wherein:

The group on Ar expanding into the unoccupied part of the ligand binding pocket is selected from the group consisting of

C₁₋₆ alkyl,

C₁₋₆ alkoxy,

C₁₋₆ alkylthio

allyloxy,

aryloxy, and

arylthio;

each of which ends in a carboxylic acid or bioisosteric replacement thereof, wherein the term “bioisosteric replacement” is defined as a substituent making interactions with PPARγ that are analogous with a COOH moiety (e.g., tetrazole, amide).

X is

a bond;

O—(CH₂)ₙ wherein n is an integer 0 to 3, e.g. O, O—CH₂, or O—(CH₂)₂;

O—(CH₂)ₙ—Y, wherein n is an integer 0 to 3, and Y is an atom selected from O, N and S, e.g.

O—(CH₂)₂—O, or O—(CH₂)₂—S;

O—(CH₂)₂—O—(CH₂)₂—NH;

O—(CH₂)₂—O(CH₂)₂—NHSO₂; or

O—(CH₂)₂—O—(CH₂)₂—NHCONH;

R is selected from the group consisting of, optionally substituted, phenyl, naphthyl, thienyl, pyridinyl, quinoxalinyl, benzoylphenyl, thiazolyl, furyl, imidazolyl, oxazolyl, pyrazinyl, quinolinyl, indolyl, benzofuran, benzothiophenyl (benzothienyl), pyrimidinyl, benzodioxolyl;

R is independently substituted in one or more positions with

C₁₋₆-alkyl,

C₁₋₆-alkoxy,

C₁₋₆-alkylthio,

C₁₋₆-acyl,

cyano,

nitro,

hydroxy,

methylhydroxy,

carboxy,

fluoromethyl,

difluoromethyl,

trifluoromethyl,

difluoromethoxy,

trifluoromethoxy,

difluoromethylthio,

trifluoromethylthio,

halogen,

formyl,

amino,

C₁₋₆-alkylamino,

di(C₁₋₆-alkyl)amino or C₁₋₆-acylamino,

aryl,

aryloxy,

arylthio,

C₁₋₆-alkylsulphonyl,

C₂₋₆-allyloxy,

benzyloxy,

benzoyl.

In particular, R can be independently substituted in one or more positions with

methyl,

ethyl,

isopropyl,

methoxy,

thiomethoxy

ethoxy,

methylsulfonyl,

formyl,

acetyl,

nitro,

cyano,

methylhydroxy,

methylamino,

carboxy,

trifluoromethyl,

trifluoromethoxy,

chloro,

fluoro,

bromo,

iodo,

benzyloxy,

amino,

dimethylamino,

acetylamino,

phenyl,

phenoxy, or

benzoyl.

The term “C₁₋₆ alkyl” denotes a straight or branched alkyl group having from 1 to 6 carbon atoms. Examples of said C₁₋₆ alkyl include methyl, ethyl, n-propyl, iso-propyl, n-butyl, iso-butyl, sec-butyl, t-butyl and straight- and branched-chain pentyl and hexyl.

The term “C₁₋₆ alkoxy” denotes a straight or branched alkoxy group having from 1 to 6 carbon atoms. Examples of said C₁₆ alkoxy include methoxy, ethoxy, n-propoxy, iso-propoxy, n-butoxy, iso-butoxy, sec-butoxy, t-butoxy and straight- and branched-chain pentoxy and hexoxy.

The term “halogen” shall mean fluorine, chlorine, bromine or iodine.

The term “aryl” denotes aromatic rings (monocyclic or bicyclic) having from 6 to 10 ring carbon atoms. Examples of said aryl include phenyl, indenyl and naphthyl.

The term “heteroaryl” denotes a mono- or bicyclic ring system (only one ring need to be aromatic, and substitution may be in any ring) having from 5 to 10 ring atoms (which are carbon atoms), in which one or more of the carbon ring atoms are other than carbon, such as nitrogen, oxygen and sulfur. Examples of said heteroaryl include pyrrole, thiazole, imidazole, thiophene, furan, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, quinazolineindole, indole, isoindole, isoindoline, indoline, benzothiophene, benzofuran, 2,3-dihydrobenzofuran, isobenzofuran, benzoxazole, 2,1,3-benzoxadiazole, benzothiazole, 2,1,3-benzothiadiazole, 2,1,3-benzoselenadiazole, benzimidazole, indazole, 2,3-dihydro-1,4-benzodioxine, indane, 1,3-benzodioxole, 3,4-dihydro-2H-1,4-benzoxazine, 1,5-naphtyridine, 1,8-naphtyridine, 1,5-naphthyridine, and 1,8-naphthyridine.

The term “heteroalkyl chain” denotes a straight or branched, saturated or unsaturated, chain comprising from 1 to 4 carbon atoms and from 1 to 4 heteroatoms selected from the group consisting of O, N, and S. The heteroatom(s) may be placed at any position of the heteroalkyl group.

Depending on the process conditions, the end products of the Formula I are obtained either in neutral or salt form (e.g., lithium, sodium, potassium salts, hydrochloride, hydrobromide, and the like).

The invention relates to a crystal of a protein-ligand complex comprising a protein-ligand complex of PPARγ and a ligand (e.g., lithium 2-[2,4-dichlorobenzoyl)amino]-5-(2-thienylmethoxy)benzoate), wherein the crystal effectively diffracts X-rays for the determination of the atomic coordinates of the protein-ligand complex to a resolution of greater (meaning better as used in this context throughout) than 5.0 Angstroms, alternatively greater than 3.0 Angstroms, or alternatively greater than 2.0 Angstroms.

One embodiment is the crystal of described above, wherein the PPARγ comprises an amino acid sequence containing amino acids amino acids Gly284, Cys285, Arg288, Ile326, Met329, Leu330, Leu333, Ile341, and Ser342 of SEQ ID NO: 1, or an amino acid sequence that differs from the amino acid sequence by only conservative substitutions, or alternatively, wherein PPARγ ligand binding domain comprises the binding site as defined herein.

This invention also features a method of using the protein-ligand crystals described herein for identifying a compound that binds to a PPARγ ligand binding domain. The method includes the steps of:

(i) using the atomic coordinates according to FIG. 1 to generate a three-dimensional structure comprising a PPARγ ligand binding domain;

(ii) employing the three-dimensional structure to identify a compound; and

(iii) determining whether the compound binds to the PPARγ ligand binding domain;

wherein the compound comprises:

(a) a benzoate group wherein the aromatic ring is capable of interacting with the side chains of Ile341 and Cys285 of SEQ ID NO: 1 and the back bone atoms of Gly284 and Cys285 of SEQ ID NO: 1;

(b) a carboxylate group bound to the benzoate group of (a), said carboxylate moiety being capable of interacting with the backbone amide nitrogen of residue Ser342 of SEQ ID NO: 1; and

(c) an aromatic group bound by an amide group to the benzoate group of (a), the said aromatic group being located in a hydrophobic region and being capable of interacting with the side chains of Leu330, Ile326, Arg288, Leu333 and Met329 of SEQ ID NO: 1.

Assays to determine if a compound binds to the PPARγ ligand binding domain are well known in the art and are also illustrated in the examples below.

This invention further features a method of using the three-dimensional structure coordinates according to FIG. 1 or Table 1, comprising:

(a) determining structure factors from the coordinates; and

(b) applying said structure factor information to a set of X-ray diffraction data obtained from a complex of another ligand and PPARγ.

In one embodiment, the invention relates to a computer-readable data storage medium comprising a data storage material encoded with computer readable data, which when used by a computer programmed with instructions for using such data, displays a three-dimensional graphical representation of a molecule or molecular complex comprising a ligand binding domain defined by structure coordinates according to FIG. 1 or Table 1, or a homologue of said molecule or molecular complex, wherein said homologue comprises a binding domain that has a root mean square deviation from the backbone atoms of said amino acids of SEQ ID NO: 1 less than about 1.50 Å, or alternatively less than about 1.00 Å.

The computer may comprise a central processing unit, a working memory, for example, random access memory and/or storage memory in the form of one or more disk drives (e.g., floppy, Zip™, Jazz™), tape drives, CD-ROM drives, DVD drives, and the like, a display terminal such as for example, a cathode ray tube type or a liquid crystal type display, and input and output lines for data transmission, including a keyboard and/or mouse controller. The computer may be a stand-alone, or connected to a network and/or shared server. Data storage materials include, for example, hard drives, floppy, Zip™ and Jazz™ type disks, tapes, CDs, and DVDs.

In another embodiment, the invention relates to a computer readable data storage material encoded with computer readable data comprising structure coordinates according to FIG. 1 or Table 1.

Alternate embodiments of the invention are those crystals described above, and methods of using such crystals or structure coordinates thereof.

Crystals of PPARγ protein or protein-ligand complex can be produced or grown by a number of techniques including batch crystallization, vapor diffusion (either by sitting drop or hanging drop), soaking, and by microdialysis. Seeding of the crystals in some instances is required to obtain X-ray quality crystals. Standard micro and/or macro seeding of crystals may therefore be used. Preferably, the crystal effectively diffracts X-rays for the determination of the atomic coordinates of the protein-ligand complex to a resolution greater than 5.0 Angstroms, alternatively greater than 3.0 Angstroms, or alternatively greater than 2.0 Angstroms. Once a crystal is produced, X-ray diffraction data can be collected. The example below used standard cryogenic conditions for such X-ray diffraction data collection though alternative methods may also be used. For example, diffraction data can be collected by using X-rays produced in a conventional source (such as a sealed tube or rotating anode) or using a synchrotron source. Methods of X-ray data collection include, but are not limited to, precession photography, oscillation photography and diffractometer data collection. Data can be processed using packages including, for example, DENZO and SCALPACK (Z. Otwinowski and W. Minor) and the like.

The three-dimensional structure of the protein or protein-ligand complex constituting the crystal may be determined by conventional means as described herein. Where appropriate, the structure factors from the three-dimensional structure coordinates of a related protein may be utilized to aid the structure determination of the protein-ligand complex. Structure factors are mathematical expressions derived from three-dimensional structure coordinates of a molecule. These mathematical expressions include, for example, amplitude and phase information. The term “structure factors” is known to those of ordinary skill in the art. Alternatively, the three-dimensional structure of the protein-ligand complex may be determined using molecular replacement analysis. This analysis utilizes a known three-dimensional structure as a search model to determine the structure of a closely related protein-ligand complex. The measured X-ray diffraction intensities of the crystal are compared with the computed structure factors of the search model to determine the position and orientation of the protein in the protein-ligand complex crystal. Computer programs that can be used in such analyses include, for example, X-PLOR and AmoRe (J. Navaza, Acta Crystallographics ASO, 157-163 (1994)). Once the position and orientation are known, an electron density map may be calculated using the search model to provide X-ray phases. The electron density can be inspected for structural differences and the search model may be modified to conform to the new structure. Using this approach, one may use the structure of the protein-ligand complex or complexes described herein to solve other protein-ligand complex crystal structures, particularly where the ligand is a different compound. Computer programs that can be used in such analyses include, for example, QUANTA and the like.

Upon determination of the three-dimensional structure of a crystal of a protein-ligand complex, a potential compound that binds to the protein (e.g., PPARγ) may be evaluated by any of several methods, alone or in combination. Such evaluation may utilize visual inspection of a three-dimensional representation of the active site, based on the X-ray coordinates of a crystal described herein, on a computer screen. Evaluation, or modeling, may be accomplished through the use of computer modeling techniques (including CADD methods), hardware, and software known to those of ordinary skill in the art. This may additionally involve model building, model docking, or other analysis of protein-ligand interactions using software including, for example, QUANTA or SYBYL, followed by energy minimization and molecular dynamics with standard molecular mechanics forcefields including, for example, CHARMM and AMBER. The three-dimensional structural information of a protein-ligand complex may also be utilized in conjunction with computer modeling to generate computer models of other protein-ligand complexes. Using the structure coordinates described herein, computer models of PPARγ-ligand (e.g., lithium 2-[(2,4-dichlorobenzoyl)amino]-5-(thienylmethoxy]benzoate), may be created using standard methods and techniques known to those of ordinary skill in the art, including software packages described herein.

Once the three-dimensional structure of a crystal comprising a protein-ligand complex formed between a protein and a standard ligand for that protein is determined, a potential ligand is examined through the use of computer modeling using a docking program such as FLEX X, DOCK, or AUTODOCK (see, Dunbrack et al., Folding & Design, 2:R27-42 (1997)), to identify potential ligands for the protein. This procedure can include computer fitting of potential ligands to the ligand binding site to ascertain how well the shape and the chemical structure of the potential ligand will complement the binding site. [Bugg et al., Scientific American, December:92-98 (1993); West et al., TIPS, 16:67-74 (1995)]. Computer programs can also be employed to estimate the attraction, repulsion, and steric hindrance of the two binding partners (i.e., the ligand-binding site and the potential ligand). Generally the tighter the fit, the lower the steric hindrances, and the greater the attractive forces, the more potent the potential drug since these properties are consistent with a tighter binding constant. Furthermore, the more specificity in the design of a potential drug, the more likely that the drug will not interact as well with other proteins. This will minimize potential side-effects due to unwanted interactions with other proteins.

A variety of methods are available to one skilled in the art for evaluating and virtually screening molecules or chemical fragments appropriate for associating with a protein. Such association may be in a variety of forms including, for example, steric interactions, van der Waals interactions, electrostatic interactions, solvation interactions, charge interactions, covalent bonding interactions, non-covalent bonding interactions (e.g., hydrogen-bonding interactions), entropically or enthalpically favorable interactions, and the like.

Numerous computer programs are available and suitable for rational drug design and the processes of computer modeling, model building, and computationally identifying, selecting and evaluating potential inhibitors in the methods described herein. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), UNITY (available from Tripos, St. Louis. Mo.), and CATALYST (available from Molecular Simulations Inc., Burlington, Mass.). Potential inhibitors may also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and the like. These techniques, methods, hardware and software packages are representative and are not intended to be comprehensive listing. Other modeling techniques known in the art may also be employed in accordance with this invention. See for example, N. C. Cohen, Molecular Modeling in Drug Design, Academic Press (1996) (and references therein), and software identified at internet sites including the CAOS/CAMM Center Cheminformatics Suite at http://www.caos.kun.nl/, and the NIH Molecular Modeling Home Page at http://www.fi.muni.cz/usr/mejzlik/mirrors/molbio.info.nih.gov/modeling/software_list/.

A potential compound that binds to PPARγ and modulates PPARγ activities is selected by performing rational design with the three-dimensional structure (or structures) determined for the crystal described herein, especially in conjunction with computer modeling and methods described above. The potential compound is then obtained from commercial sources or is synthesized from readily available starting materials using standard synthetic techniques and methodologies known to those of ordinary skill in the art. The potential compound is then assayed to determine its ability to modulate PPARγ activities.

The potential compound selected or identified by the aforementioned process may be assayed to determine its ability to modulate (e.g., inhibit or stimulate) PPARγ activity. The assay may be in vitro or in vivo. Modulation can be measured by various methods, including, for example, those methods illustrated in the examples below. The compounds described herein may be used in assays, including radiolabelled, antibody detection and fluorometric, for the isolation, identification, or structural or functional characterization. The assay may be a protein inhibition assay, utilizing a full length or truncated protein, said protein having sequence homology with that of mammalian origin, including for example, human, murine, rat, and the like. The protein is contacted with the potential inhibitor and a measurement of the binding affinity of the potential inhibitor against a standard is determined. Such assays are known to one of ordinary skill in the art. The assay may also be a cell-based assay. The potential compound is contacted with a cell and a measurement of inhibition of a standard marker produced in the cell is determined. Cells may be either isolated from an animal, including a transformed cultured cell, or may be in a living animal. Such assays are also known to one of ordinary skill in the art.

When suitable potential compounds are identified as described above, a supplemental crystal can be produced or grown (using techniques described herein) that comprises a protein-ligand complex formed between a protein (e.g., PPARγ) and the potential ligand. Preferably, the crystal effectively diffracts X-rays for the determination of the atomic coordinates of the protein-ligand complex to a resolution greater than 5.0 Angstroms, alternatively greater than 3.0 Angstroms, or alternatively greater than 2.0 Angstroms. The three-dimensional structure of the protein-ligand complex constituting the supplemental crystal may be determined by conventional means such as those described herein.

The potential compound described above is selected by performing rational drug design with the three-dimensional structure (or structures) determined for the supplemental crystal, especially in conjunction with computer modeling described above. The potential compound is then obtained from commercial sources or is synthesized from readily available starting materials using standard synthetic techniques and methodologies known to those of ordinary skill in the art. The potential compound is then assayed to determine its ability to modulate PPARγ activities.

For all potential compound (agonist, antagonist) screening or assay methods described herein, further refinements to the structure of the potential compound may generally be necessary and can be made by successive iterations of any/or all of the steps provided by the screening assay method.

Once the potential compound is determined to bind to PPARγ protein and to modulate its activities, the compound may be useful in therapeutic or prophylactic treatment of mammals, including man, for conditions where modulation of either PPARγ or PPARγ activity, or the combination of both PPARγ. and PPARγ activities. Such conditions could be e.g. diabetes, diabetes mellitus type 2, insulin resistance, impaired glucose tolerance and/or in combinations with dyslipidemias, obesity, atherosclerosis, coronary artery disease, PCOS, gestational diabetes, or inflammation.

The compounds described above are particularly useful for the treatment of type II diabetes, in combination(s) with dyslipidemias, obesity, atherosclerosis and coronary artery disease. For this purpose the compounds can be used alone or in combination(s) with sulfonylureas, metformin, alpha-glycosidase inhibitors, insulin or other anti-diabetic treatments/agents. Reference to treatment is intended to include prophylaxis as well as the alleviation of established symptoms.

For clinical use, the compounds are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. The formulations can be further prepared by known methods such as granulation, compression, microencapsulation, spray coating, etc.

The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. The typical daily dose of the active substance varies within a wide range and will depend on various factors such as for example the individual requirement of each patient and the route of administration.

The compounds may also be administered as prodrugs that may be converted to the active ingredient in question after metabolic transformation in vivo. Conventional procedures for the selection and preparation of suitable prodrug derivatives are described, for example, in “Design of Prodrugs” ed. H. Bundgaard, Elsevier, 1985.

“An effective amount” refers to an amount of a compound which confers a therapeutic effect on the treated subject (eg., a human, a mammal, a horse, a dog, or a cat). The therapeutic effect may be objective (i.e., measurable by some test or marker) or subjective (i.e., subject gives an indication of or feels an effect). The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The compounds can be prepared by, or in analogy with, standard synthetic methods, and especially according to, or in analogy with, the methods described in the co-pending international patent application derived from Swedish patent application No. 0102384-5, filed Jul. 3, 2001.

More specifically, the compounds described above can be prepared by, or in analogy with, standard synthetic methods, and especially according to, or in analogy with, the following methods.

Method 1

Compounds of formula (I) in which X is oxygen can be prepared beginning with commercially available 2-amino-5-hydroxybenzoic acid (i) as shown in Scheme 1. The corresponding methyl ester (ii) is formed by treatment with sulfuric acid and methanol and is subsequently coupled with a benzoyl chloride or a heteroarylcarbonyl chloride (commercially available or prepared from the corresponding carboxylic acid using thionyl chloride or oxalyl chloride) to provide the amide (iii). Reaction of (iii) with an alcohol in the presence of diethyl azodicarboxylate (DEAD) or 1,1′-azobis(N,N-dimethylformamide) (TMAD; cf. Tetrahedron Lett. 1995, vol. 36: 3789-3792) and triphenylphosphine or polymer supported triphenylphosphine in a solvent such as dichloromethane and/or tetrahydrofuran (Mitsunobu reaction; see Org. React. 1992, vol. 42: 335-656) gives the adduct (iv). Ester hydrolysis, using 1M lithium hydroxide, affords the target compounds (v) as lithium salts.

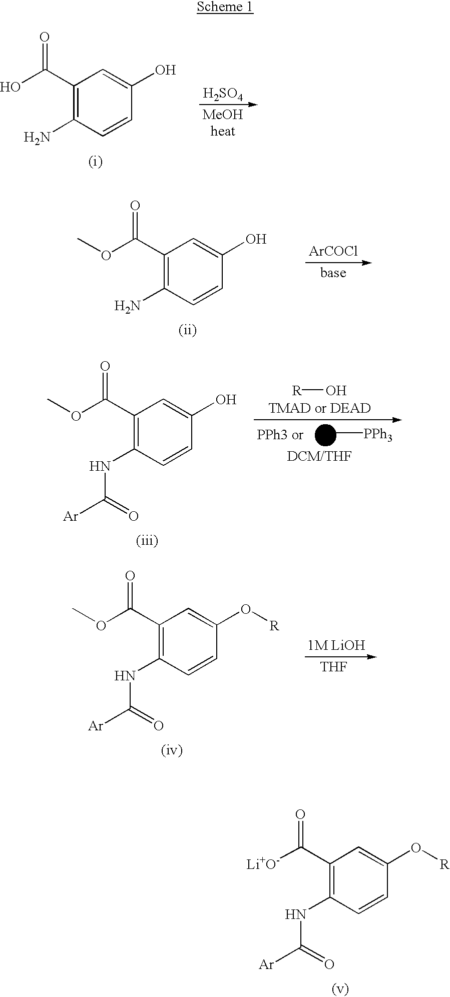

Method 2

Other compounds of the present invention can be prepared as shown in Scheme 2. The Mitsunobu reaction can also be performed on the intermediate (ii), i.e. before the amide coupling, to form the adduct (vi). Subsequent amide coupling and ester hydrolysis afford the target compounds (v).

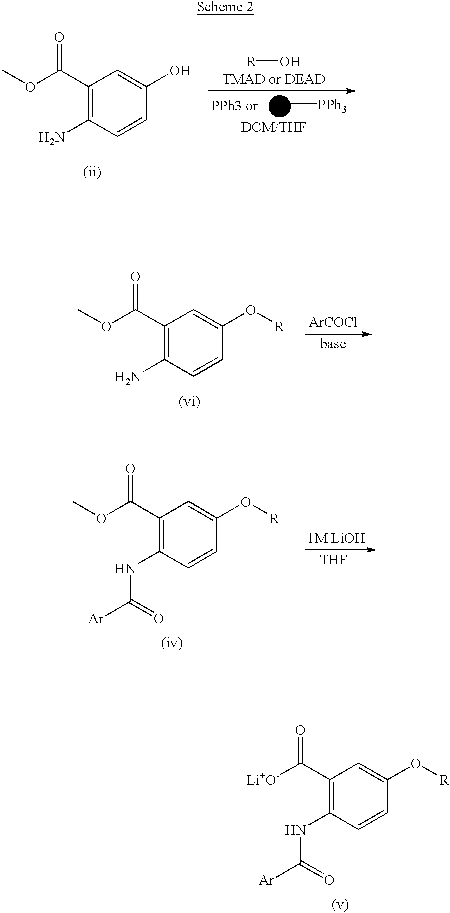

Method 3

Compounds of formula (I) in which X=C₀ and R is an aryl or heteroaryl substituent can be prepared as outlined in Scheme 3. Treatment of the commercially available 2-amino-5-iodobenzoic acid (vii) with trichloromethyl chloroformate in solvents such as dioxane gives the isatoic anhydride (viii) which can be further reacted with methanol and a base such as potassium carbonate to form the methyl ester (ix). Subsequent coupling with a benzoyl chloride or a heteroarylcarbonyl chloride (commercially available or prepared from the corresponding carboxylic acid using thionyl chloride or oxalyl chloride) provides amide (x). Palladium-catalyzed cross-coupling of (x) with an aryl or heteroaryl boronic acid (Suzuki coupling; see Chem. Rev. 1995, 95, 2457-2483) gives biaryl (xii) or a mixture of (xii) and the bicycle (xi). Subsequent ester hydrolysis using 1M lithium hydroxide solution affords the target compounds (xiii).

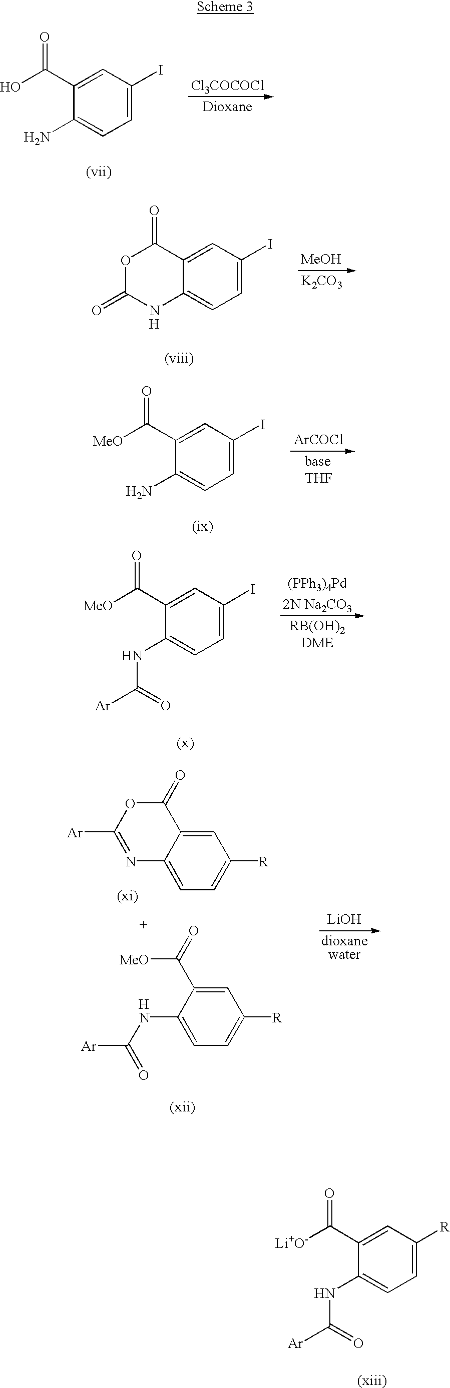

Method 4

Other compounds of the present invention can be prepared as shown in Scheme 4. The intermediate (iii) can be reacted with nitrogen containing heterocycles to form diaryl ethers (xiv) which can be hydrolyzed as described earlier to afford compounds (xv).

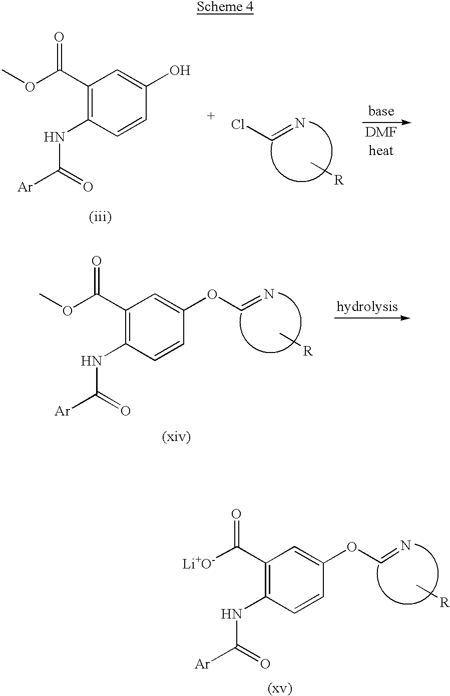

Method 5

Other compounds of the present invention can be prepared as shown in Scheme 5. Intermediate (iii) can be reacted with benzylic (or aliphatic) bromides to form compounds (xvi) which can be hydrolyzed as described earlier to afford compounds (xvii).

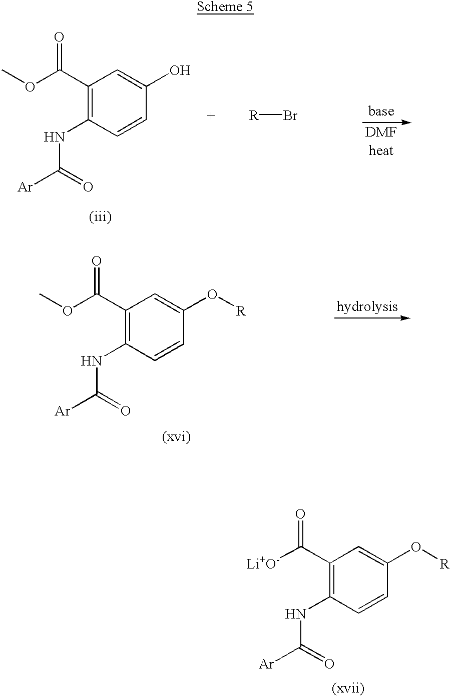

The chemicals used in the above-described synthetic routes may include, for example, solvents, reagents, catalysts, protecting group and deprotecting group reagents. The methods described above may also additionally include steps, either before or after the steps described specifically herein, to add or remove suitable protecting groups in order to ultimately allow synthesis of the compounds of Formula (I). In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. Without further elaboration, it is believed that one skilled in the art can, based on the description herein, utilize the present invention to its fullest extent. All references, documents, and publications (including patent applications, journal articles, texts, treatises, software packages, and web sites) cited herein are hereby incorporated by reference in their entirety.

### EXAMPLES

### Example 1

### Synthesis of Lithium 2-[(2,4-dichlorobenzoyl)amino]-5-(2-thienylmethoxy)benzoate

Step 1: Methyl 2-amino-5-hydroxybenzoate

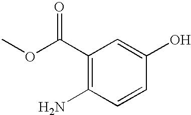

To a stirred suspension of 2-amino-5-hydroxybenzoic acid (15 g, 98 mmol) in methanol (100 ml) was added sulfuric acid (95%, 15 ml) at room temperature. The solution was stirred at 90° C. for 3.5 hours after which it was allowed to reach room temperature and carefully poured into saturated sodium bicarbonate. Subsequent extraction with chloroform (3×300 ml), drying of the organic phase using magnesium sulfate and concentration in vacuo gave the title compound (15 g, 80%) as a dark solid. mp: 154-155° C.; ¹H NMR (DMSO) δ 8.66 (s, 1H), 7.09 (d, J=2.72 Hz 1H), 6.82-6.76 (m, 1H), 6.66-6.60 (m, 1H), 6.07 (br s, 2H), 3.75 (s, 3H); ¹³C NMR (DMSO) δ 167.7, 146.6, 144.8, 123.6, 117.9, 114.4, 108.8, 51,4; MS m/z 168 (M+1).

Step 2: Methyl 2-[(2,4-dichlorobenzoyl)amino]-5-hydroxybenzoate

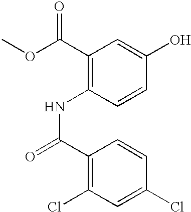

To a stirred mixture of methyl 2-amino-5-hydroxybenzoate (10 g, 60 mmol) pyridine (80 ml) and molecular sieves (4 Å), 2,4-dichlorobenzoyl chloride (7.6 ml, 54 mmol) in pyridine (3 ml) was added slowly at 0° C. The mixture was allowed to reach room temperature and then stirred over night. After addition of chloroform, the mixture was filtered and the filtrate washed with 1M hydrochloric acid (3×150 ml) and brine, dried with magnesium sulfate and concentrated in vacuo. The residue was re-crystallized from chloroform to give the title compound (4 g, 20%) as a gray solid. mp: 181-182° C.; ¹H NMR (DMSO) δ 10.64 (s, 1H), 9.81 (s, 1H), 7.92-7.55 (m, 4H), 7.29 (d, J=2.73 Hz 1H), 7.08-7.02 (m, 1H), 3.79 (m, 3H); MS m/z 338 (M−1).

Step 3: Methyl 2-[(2,4-dichlorobenzoyl)amino]-5-(2-thienylmethoxy)benzoate

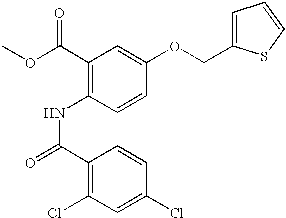

TMAD (183 mg, 1.06 mmol) was added to a suspension of methyl 2-[(2,4-dichlorobenzoyl)amino]-5-hydroxybenzoate (240 mg, 0.71 mmol; prepared in Example XX), polymer bound triphenylphosphine (480 mg, 1.4 mmol) and thiophene-2-methanol (73 μl, 0.78 mmol) in anhydrous THF (3 ml) and DCM (3 ml). The suspension was shaken at room temperature over night and filtered through a plug of Celite. The filtrate was concentrated in vacuo and the residue purified by chromatography on silica gel eluting with CHCl₃ to give the title compound (130 mg, 42%) as yellow oil. ¹H NMR (CDCl₃) δ 11.31 (s, 1H), 8.79 (d, J=9.40 Hz 1H), 7.67-7.57 (m, 2H), 7.47 (d, J=1.98 Hz 1H), 7.35-7.30 (m, 2H), 7.27-7.21 (m, 1H), 7.12-7.09 (m, 1H), 7.02-6.97 (m, 1H), 5.23 (s, 1H), 3.89 (s, 3H); ³C NMR (CDCl₃) δ 168.3, 164.1, 153.7, 138.7, 136.9, 135.2, 134.7, 132.3, 130.5, 130.4, 127.6, 127.2, 127.0, 126.6, 122.2, 122.0, 116.6, 166.6, 65.5, 52.7

Step 4: Lithium 2-[(2,4-dichlorobenzoyl)amino]-5-(2-thienylmethoxy)benzoate

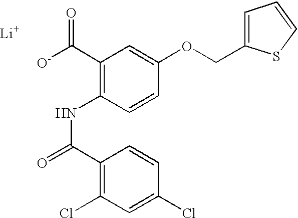

Lithium hydroxide (1M solution, 298 μl) was added at room temperature to a stirred solution of methyl 2-[(2,4-dichlorobenzoyl)amino]-5-(2-thienylmethoxy)benzoate (130 mg, 0.30 mmol) in THF (2 ml). The mixture was stirred over night and then concentrated in vacuo, re-dissolved in methanol and concentrated again. The residue was washed with diethyl ether to give the title compound (120 mg, 94%) as yellow solid. mp: 165-168° C.; ¹H NMR (CD₃OD) δ 8.56 (d, J=8.91 Hz 1H), 7.75 (d, J=2.97 Hz 1H), 7.66-7.56 (m, 2H), 7.47-7.37 (m, 2H), 7.17-7.13 (m, 1H), 7.08 (dd, J=9.16, 3.22 Hz 1H), 7.02-6.97 (m, 1H), 5.27 (s, 2H); ¹³C NMR (CD₃OD) δ 172.5, 164.4, 154.1, 139.6, 136.2, 135.6, 133.6, 132.1, 129.9, 127.4, 126.7, 126.3, 125.9, 125.6, 120.6, 118.0, 116.9, 64.9; MS m/z 420 (M−1).

### Example 2

### Crystal Structure of the PPARγ Ligand Binding Domain in Complex With Lithium 2-[(2,4-dichlorobenzoyl)amino]-5-(2-thienylmethoxy)benzoate.

The structure of the PPARγ ligand-binding domain in complex with lithium 2-[(2,4-dichlorobenzoyl)amino]-5-(2-thienylmethoxy)benzoate (Example 1) was determined by X-ray crystallography. Standard molecular biology techniques were used to produce the ligand binding domain of human PPARγ in E. Coli bacterial cells. The protein was purified to homogeneity and concentrated to a final concentration of 10 mg/ml. Crystals of PPARγ-LBD complexes were grown by the hanging drop diffusion method at 18° C. and appeared in 3-5 days. The well solution contained 0.1M Tris/HCl buffer, pH 7.5, 22% polyethylene glycol 3000 and 0.2M Ca acetate. Typically 3 μl of the precipitant was mixed with 3 μl of a solution containing 9 mg/ml PPARγ-LBD, 1 mM GRIP-1 co-activator peptide (KEKHKILHRLLQDS, SEQ ID NO: 2) and 1 mM ligand in the drop. Crystals were mounted in glass capillaries and diffracted to 2.90 Å. All data were collected at room temperature using a Rigaku RU300 rotating anode with Molecular Structure Corp. mirrors and an Raxis4 image plate detector. The data were processed with the programs DENZO and Scalepack. The structures were solved by molecular replacement with coordinates from PDB entry 3prg, using the AMoRe program package. Model building was performed using the O software package, and the models were refined using simulated annealing and restrained B factor refinement included in CNS. The ligand in example 1 and co-activator peptide were modeled according to the difference electron density maps.

The obtained crystal structure indicated a new binding mode for a PPARγ ligand. In contrast to the binding mode previously suggested by Nolte et al. (1998; Nature 395: 137-143), the novel binding mode positions the ligand in a region distant from helix 12 and the residues His323, His449 and Tyr473 of SEQ ID NO: 1. The novel binding mode is characterized by:

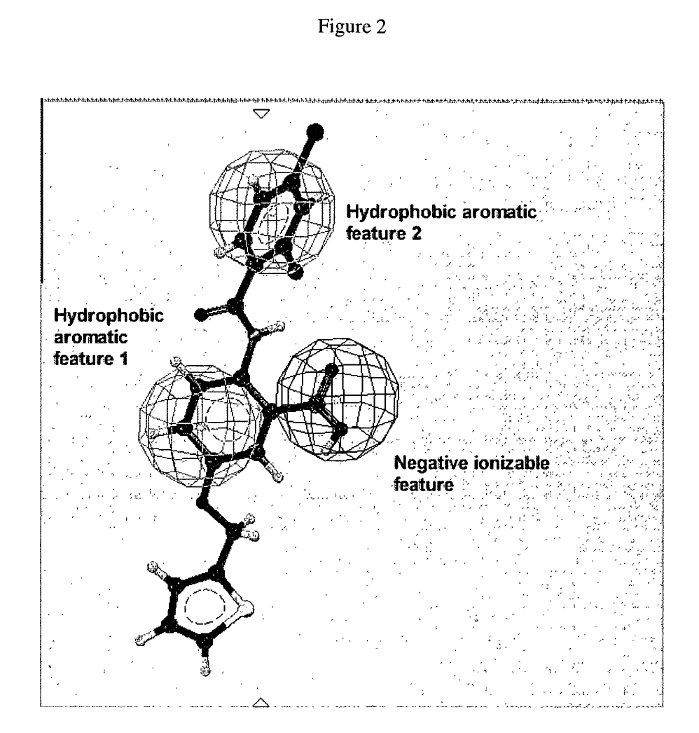

(a) There is a polar interaction between the carboxylate moiety (designated “negative ionizing feature” in FIG. 2) of the 5-substituted 2-amidobenzoic acid ligand and the backbone amide nitrogen of residue Ser342 of SEQ ID NO: 1 (FIG. 1). The carboxylate group is further stabilized by a hydrogen bond to the amide nitrogen in the ligand.

(b) The aromatic ring of the benzoate group (designated “hydrophobic aromatic feature 1” in FIG. 2) is interacting through the side chains of Ile341 and Cys285 of SEQ ID NO: 1 and the backbone atoms of Gly284 and Cys285 of SEQ ID NO: 1.

(c) An additional aromatic group is linked by an amide to the benzoate group. This aromatic group (designated “hydrophobic aromatic feature 2” in FIG. 2) is located in a hydrophobic region and interacts with the side chains of Leu330, Ile326, Arg288, Leu333 and Met329 of SEQ ID NO: 1.

The interacting features in the pharmacophore, defined by the X-ray structure and extracted from the ligand coordinates using the Catalyst software (Accelrys), are illustrated in FIG. 1 and the coordinates are given in TABLE 2.

### Example 3

### Ligand Binding Assay

Crude extracts are prepared from E. coli (BL21(DE3)pLysS, Novagen) producing GST-PPARγLBD fusion protein by freeze thawing in buffer containing 50 mM Tris-HCl pH 7.9, 250 mM KCl, 10% glycerol, 1% Triton X-100, 10 mM DTT, 1 mM PMSF, 10 μg/mL DNase and 10 mM MgCl. Competitive ligand binding assays are performed on immobilized GST-GST-PPARγLBD fusion protein from crude extracts incubated with glutathione-Sepharose 4B (Amersham Pharmacia Biotech). Following immobilization, the slurry is washed three times in binding buffer containing 50 mM Tris-HCL, pH 7.9, 50 mM KCl, 0.1% Triton-X100, 10 mM DTT, 2 mM EDTA, dispensed in 96-well filter plates (MHVB N45, Millipore) and incubated with a fixed amount tritiated ligand and different concentrations of cold competing ligands. Equilibrium binding is reached after incubation for 2 hours at room temperature on a plate shaker. The plates are then washed 3 times in binding buffer, dried overnight at room temperature followed by scintillation counting after the addition of 25 μl of scintillant (Optiscint Hisafe, Wallac) per well. Each experiment is performed in duplicate and repeated independently at least three times. ³-BRL49653 (ART-605; American Radiolabeled Chemicals, USA) is used as tracer in PPARγ competitive ligand binding experiments at a concentration of 30 nM. The compounds of Formula I exhibit Kᵢ values on PPARγ in the range of 0.3 to 35 μM.

### Example 4

### Cell-Based Reporter Assay

The effect of identified compounds on activation of PPARγ is determined. Reporter gene assays are performed essentially as described in Bertilsson et al., 1998 (Proc. Natl. Acad. Sci. U.S.A. 95:12208-12213), by transient co-transfections of CaCo2/TC cells with a GAL-4-LBD (Ligand Binding Domain) fusion constructs, containing the nucleotide sequence corresponding to human PPARγLBD (i.e. amino acid residues 204-477), together with a 4×UAS-luciferase reporter gene construct, using the FuGENE-6 transfection reagent (Roche) according to the manufacturers recommendations. After 24 hours, the cells are treated with trypsin, transferred to 96-well microplates and allowed to settle. Induction is performed for 24 hours by applying different concentrations of compounds diluted in DMSO or DMSO alone (vehicle). Subsequently, the cells are lysed and luciferase activity measured, according to standard procedures. The compounds of Formula I exhibit EC₅₀ values on PPARγ in the range of 0.3 to 50 μM.

What is claimed is:

1. A method for identifying a compound capable of selectively modulating the activity of PPARγ, said method comprising:
(i) providing test compounds that are selected to fit spatially and preferentially into a PPARγ ligand binding domain;
(ii) screening said test compounds for binding to the PPARγ ligand binding domain; and
(iii) identifying a test compound that selectively modulates the activity of PPARγ;
wherein said compound comprises:
(a) a benzoate group wherein the aromatic ring is capable of interacting with the side chains of Ile341 and Cys285 of SEQ ID NO: 1 and the back bone atoms of Gly284 and Cys285 of SEQ ID NO: 1;
(b) a carboxylate group bound to the benzoate group of (a), said carboxylate moiety being capable of interacting with the backbone amide nitrogen of residue Ser342 of SEQ ID NO: 1; and
(c) an aromatic group bound by an amide group to the benzoate group of (a), the said aromatic group being located in a hydrophobic region and being capable of interacting with the side chains of Leu330, Ile326, Arg288, Leu333 and Met329 of SEQ ID NO: 1.

2. The method according to claim 1 wherein the PPARγ ligand binding domain is based on a structural model of PPARγ bound to the compound lithium 2-[(2,4-dichlorobenzoyl)amino]-5-(2-thienylmethoxy)benzoate.

3. The method of claim 2 wherein the PPARγ ligand binding domain has the pharmacophoric features shown in TABLE 2.

4. The method according to claim 1 wherein the PPARγ ligand binding domain is defined by atomic coordinates of amino acids Gly284, Cys285, Arg288, Ile326, Met329, Leu330, Leu333, Ile341, and Ser342 of SEQ ID NO: 1 according to FIG. 1.

5. The method according to claim 1 wherein the test compound is selected by structure-based design.

6. The method according to claim 1 wherein the test compound is selected by virtual screening of compound databases.

7. The method of claim 1 wherein said compound is an agonist of PPARγ.

8. The method of claim 1 wherein said compound is of the Formula I: 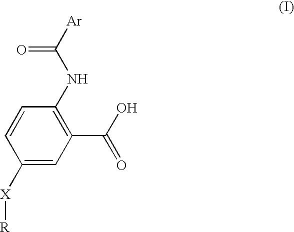
or a pharmaceutically acceptable salt or a prodrug form thereof, wherein:
Ar is an 5- or 6-membered aromatic group or a fused aromatic ring system, substituted with a group expanding into the unoccupied part of the ligand binding pocket and making hydrogen bonding interactions with one or more of the side chain of Tyr473, His323 and His449 of SEQ ID NO: 1;
X is
a bond, or
a heteroalkyl chain comprising from 1 to 4 carbon atoms and from 1 to 4 heteroatoms, or
a formula 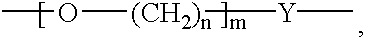
wherein m is 0, 1, or 2,
n is 0, 1, 2, or 3, and
Y is a bond, O, S, NH, NHSO₂, NHC(O)NH, or CH═CH; and
R is an optionally substituted aryl or heteroaryl group.

9. The method of claim 8 wherein the group on Ar expanding into the unoccupied part of the ligand binding pocket is selected from the group consisting of
C₁₋₆ alkyl,
C₁₋₆ alkoxy,
C₁₋₆ alkylthio,
allyloxy,
aryloxy, and
arylthio;
each of which ends in a carboxylic acid or bioisosteric replacement thereof, defined as making analogous interactions with the PPARγ as the COOH moiety.

10. A compound identified by the method according to claim 1, wherein the compound is an agonist of PPARγ.

11. A pharmaceutical composition comprising a compound identified by the method according to claim 1, in an amount effective for treating or preventing diabetes and a pharmaceutically acceptable carrier, wherein the compound is an agonist of PPARγ.

12. A method for identifying a compound that binds to a PPARγ ligand binding domain, comprising the steps of:
(i) using the atomic coordinates according to FIG. 1 to generate a three-dimensional structure comprising a PPARγ ligand binding domain;
(ii) employing the three-dimensional structure to identify a compound; and
(iii) determining whether the compound binds to the PPARγ ligand binding domain;
wherein the compound comprises:
(a) a benzoate group wherein the aromatic ring is capable of interacting with the side chains of Ile341 and Cys285 of SEQ ID NO: 1 and the back bone atoms of Gly284 and Cys285 of SEQ ID NO: 1;
(b) a carboxylate group bound to the benzoate group of (a), said carboxylate moiety being capable of interacting with the backbone amide nitrogen of residue Ser342 of SEQ ID NO: 1; and
(c) an aromatic group bound by an amide group to the benzoate group of (a), the said aromatic group being located in a hydrophobic region and being capable of interacting with the side chains of Leu330, Ile326, Arg288, Leu333 and Met329 of SEQ ID NO: 1.

13. The method according to claim 12, wherein the compound is identified by structure-based design.

14. The method according to claim 12, wherein the compound is identified by virtual screening of compound databases.

15. The method according to claim 12, wherein the compound is identified by computer assisted drug design.

16. The method according to claim 12, wherein the compound is a compound of formula I as defined in claim 8.

17. A computer-readable data storage medium comprising a data storage material encoded with computer readable data, which when used by a computer programmed with instructions for using such data, displays a three-dimensional graphical representation of a molecule or molecular complex comprising a ligand binding domain defined by structure coordinates according to FIG. 1.

18. The computer-readable data storage medium of claim 17, wherein the ligand binding domain includes amino acids Gly284, Cys285, Arg288, Ile326, Met329, Leu330, Leu333, Ile341, and Ser342 of SEQ ID NO: 1.

19. A method for identifying a compound capable of selectively modulating the activity of PPARγ, said method comprising:
(i) providing test compounds that fit spatially and preferentially into a PPARγ ligand binding domain;
(ii) screening said test compounds for binding to the PPARγ ligand binding domain; and
(iii) identifying a test compound that selectively modulates the activity of PPARγ;
wherein said compound comprises:
(a) a benzoate group wherein the aromatic ring is capable of interacting with the side chains of Ile341 and Cys285 of SEQ ID NO: 1 and the back bone atoms of Gly284 and Cys285 of SEQ ID NO: 1;
(b) a carboxylate group bound to the benzoate group of (a), said carboxylate moiety being capable of interacting with the backbone amide nitrogen of residue Ser342 of SEQ ID NO: 1; and
(c) an aromatic group bound by an amide group to the benzoate group of (a), the said aromatic group being located in a hydrophobic region and being capable of interacting with the side chains of Leu330, Ile326, Arg288, Leu333 and Met329 of SEQ ID NO: 1.

20. The method of claim 19, wherein the PPARγ ligand binding domain is that of PPARγ when bound with a ligand of formula I as defined in claim 8.

21. The method according to claim 19 wherein the PPARγ ligand binding domain is based on a structural model of PPARγ bound to the compound lithium 2-[(2,4-dichlorobenzoyl)amino]-5-(2-thienylmethoxy)benzoate.
